(19) United States
(12) Patent Application Publication
(10) Pub. No.: US 2011/0244049 A1
(43) Pub. Date: Oct. 6, 2011

(54) COMPOSITIONS COMPRISING 4-(2-(5-BROMO-4-(1-CYCLOPROPYLNAPHTHALEN-4-YL)-4H-1,2,4-TRIAZOL-3-YLTHIO)ACETAMIDO)-3-CHLOROBENZOIC ACID AND PHARMACEUTICALLY ACCEPTABLE SALTS THEREOF, AND METHODS FOR PREPARING AND USING SAME

(75) Inventors: Barry D. Quart, Encinitas, CA (US); Colin E. Rowlings, Encinitas, CA (US)

(73) Assignee: ARDEA BIOSCIENCES INC., SAN DIEGO, CA (US)

(21) Appl. No.: 13/125,697

(22) PCT Filed: Oct. 23, 2009
(86) PCT No.: PCT/US09/61970

§ 371 Date: Apr. 28, 2011

(30) Foreign Application Priority Data
Oct. 24, 2008 (US) 61108437

(51) Int. Cl. A61K 31/4196; A61K 9/16; A61P 31/18

(52) U.S. Cl. 424/494; 514/384; 424/501; 424/499

(57) ABSTRACT

The present invention relates to compositions comprising 4-(2-(5-bromo-4-(1-cyclopropylnaphthalen-4-yl)-4H-1,2,4-triazol-3-ylthio)acetamido)-3-chlorobenzoic acid or pharmaceutically acceptable salts thereof, and to the preparation and use of such compositions, in particular for the treatment of diseases.

### RELATED APPLICATION

This application claims priority to U.S. Provisional Application No. 61/108,437 filed Oct. 24, 2008, which is incorporated herein by reference in its entirety.

### BACKGROUND OF THE INVENTION

4-(2-(5-Bromo-4-(1-cyclopropylnaphthalen-4-yl)-4H-1,2,4-triazol-3-ylthio)acetamido)-3-chlorobenzoic acid and pharmaceutically acceptable salts thereof, are useful in the treatment and prevention of diseases. Described herein are compositions comprising 4-(2-(5-bromo-4-(1-cyclopropylnaphthalen-4-yl)-4H-1,2,4-triazol-3-ylthio)acetamido)-3-chlorobenzoic acid and pharmaceutically acceptable salts thereof and methods of using the compositions, for example in the treatment of diseases.

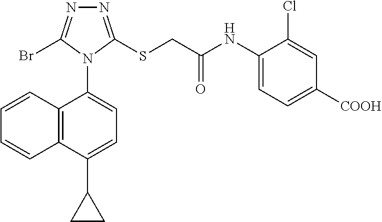

### SUMMARY OF THE INVENTION

Disclosed herein, in certain embodiments, is a composition comprises greater than about 35% by weight of a compound or mixture of compounds of structure (I):

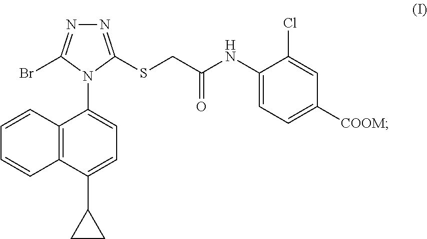

wherein M is hydrogen, sodium, potassium, calcium or arginine. In some embodiments, the composition comprises greater than about 60% by weight of a compound or mixture of compounds of structure (I). In some embodiments, the composition comprises greater than about 70% by weight of a compound or mixture of compounds of structure (I). In some embodiments, the composition comprises greater than about 80% by weight of a compound or mixture of compounds of structure (I). In some embodiments, the composition comprises at least 100 mg of a compound or mixture of compounds of structure (I). In some embodiments, the composition comprises at least 250 mg of a compound or mixture of compounds of structure (I). In some embodiments, the composition comprises at least 500 mg of a compound or mixture of compounds of structure (I). In some embodiments, the composition comprises at least 750 mg of a compound or mixture of compounds of structure (I). In some embodiments, the composition comprises at least 800 mg of a compound or mixture of compounds of structure (I). In some embodiments, the composition comprises about 100 mg, about 200 mg, about 300 mg, about 400 mg, about 500 mg, about 600 mg, about 700 mg, about 800 mg, about 900 mg or about 1000 mg of a compound or mixture of compounds of structure (I). In some embodiments, the composition comprises about 600 mg, about 800 mg or about 1000 mg of a compound or mixture of compounds of structure (I). In some embodiments, the composition comprises about 600 mg of a compound or mixture of compounds of structure (I). In some embodiments, the composition comprises about 800 mg of a compound or mixture of compounds of structure (I). In some embodiments, the composition comprises about 1000 mg of a compound or mixture of compounds of structure (I). In some embodiments, the composition further comprises one or more pharmaceutically acceptable diluents, binders, lubricants, coating agents, barrier coatings, plasticizers, dispersing agents or film coating additives. In some embodiments, the composition further comprises one or more pharmaceutically acceptable diluents. The composition of claim [0003], wherein the diluent is microcrystalline cellulose, silicified microcrystalline cellulose, cellulose, lactose, compressible sugar, mannitol, calcium silicate and calcium phosphate, sodium phosphate, sodium carbonate, or a combination thereof. In some embodiments, the diluent is in powder form. In some embodiments, the diluent is in granular form. In some embodiments, the diluent is microcrystalline cellulose. In some embodiments, the composition comprises one or more pharmaceutically acceptable binders. In some embodiments, the binder is hypromellose, povidone, hydroxypropyl cellulose, hydroxyethyl cellulose or starch. In some embodiments, the binder is hypromellose. In some embodiments, the composition further comprises one or more pharmaceutically acceptable lubricant. In some embodiments, the lubricant is magnesium stearate, stearic acid or sodium stearyl fumarate. In some embodiments, the lubricant is magnesium stearate. In some embodiments, the composition further comprises one or more pharmaceutically acceptable barrier coatings. In some embodiments, the pharmaceutically acceptable barrier coating is a pharmaceutically acceptable enteric coating. In some embodiments, the he barrier coating is hypromellose or polyvinyl alcohol. In some embodiments, the barrier coating is hypromellose. In some embodiments, the composition further comprises one or more pharmaceutically acceptable coating agents. In some embodiments, the coating agent is a methacrylic acid based copolymer, Eudragit L30D55, Acryl-Eze, hydroxypropylmethyl cellulose acetate succinate, polyvinylacetate phthalate, cellulose acetate phthalate or a combination thereof. In some embodiments, the coating agent is a methacrylic acid based copolymer, Eudragit L30D55 or Acryl-Eze. In some embodiments, the composition further comprises one or more pharmaceutically acceptable plasticizers. In some embodiments, the plasticizer is triethyl citrate, triacetin, dibutyl phthalate, diethyl phthalate or glycerin. In some embodiments, the plasticizer is triethyl citrate. In some embodiments, the composition further comprises one or more pharmaceutically acceptable film coating additives. In some embodiments, the film coating additive is talc, glycerol monostearate or colloidal silicon dioxide. In some embodiments, the film coating additive is talc. In some embodiments, the composition further comprises one or more pharmaceutically active compounds. In some embodiments, at least one of the one or more pharmaceutically active compounds has anti-viral activity In some embodiments, the anti-viral activity is anti-HIV or anti-AIDS activity. In some embodiments, the composition further comprises two pharmaceutically active compounds. In some embodiments, at least one of the two pharmaceutically active compounds has anti-viral activity. In some embodiments, the anti-viral activity is anti-HIV or anti-AIDS activity. In some embodiments, the composition is a unitary dosage form. In some embodiments, M is hydrogen or potassium. In some embodiments, M is potassium. In some embodiments, the composition is suitable for oral administration to a mammal. In some embodiments, the composition is suitable for oral administration to a human.

Disclosed herein, in certain embodiments, is a composition comprises: a compound of structure (I):

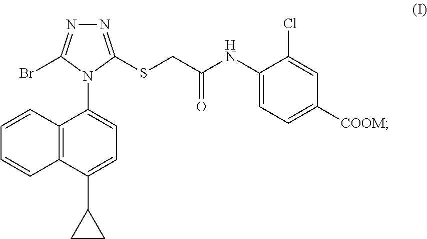

wherein M is hydrogen, sodium, potassium, calcium, or arginine; and one or more diluents; one or more binders; one or more coating agents; one or more dispersing agents; and one or more plasticizers.

In some embodiments, the composition is a unitary dosage form. In some embodiments, the composition is encapsulated. In some embodiments, the composition is encapsulated within a hard gelatin capsule. In some embodiments, M is hydrogen or potassium. In some embodiments, M is potassium. In some embodiments, the diluent is microcrystalline cellulose, silicified microcrystalline cellulose, cellulose, lactose, compressible sugar, mannitol, calcium silicate and calcium phosphate in powder and granular forms, sodium phosphate or sodium carbonate. In some embodiments, the diluent is microcrystalline cellulose. In some embodiments, the binder is hypromellose, povidone, hydroxypropyl cellulose, hydroxyethyl cellulose or starch. In some embodiments, the binder is hypromellose. In some embodiments, the coating agent is a methacrylic acid based copolymers, Eudragit L30D55, Acryl-Eze, hydroxypropylmethyl cellulose acetate succinate, polyvinylacetate phthalate or cellulose acetate phthalate. In some embodiments, the coating agent is a methacrylic acid based copolymer. In some embodiments, the dispersing agent is talc, glycerol monostearate or colloidal silicon dioxide. In some embodiments, the dispersing agent is talc. In some embodiments, the plasticizer is triethyl citrate, triacetin, dibutyl phthalate, diethyl phthalate or glycerin. In some embodiments, the plasticizer is triethyl citrate. In some embodiments, the composition comprises from about 1 mg to about 1000 mg of the compound of structure (I). In some embodiments, the composition comprises from about 10 mg to about 1000 mg of the compound of structure (I). In some embodiments, the composition comprises from about 50 mg to about 1000 mg of the compound of structure (I). In some embodiments, the composition comprises about 100 mg of the compound of structure (I). In some embodiments, the composition comprises about 200 mg of the compound of structure (I). In some embodiments, the composition comprises about 300 mg of the compound of structure (I). In some embodiments, the composition comprises about 400 mg of the compound of structure (I). In some embodiments, the composition comprises about 500 mg of the compound of structure (I). In some embodiments, the composition comprises about 600 mg of the compound of structure (I). In some embodiments, the composition comprises about 700 mg of the compound of structure (I). In some embodiments, the composition comprises about 800 mg of the compound of structure (I). In some embodiments, the composition comprises about 900 mg of the compound of structure (I In some embodiments, the composition comprises about 1000 mg of the compound of structure (I). In some embodiments, the composition comprises from about 10% to about 90% by weight of the compound of structure (I). In some embodiments, the composition comprises from about 25% to about 90% by weight of the compound of structure (I). In some embodiments, the composition comprises from about 50% to about 90% by weight of the compound of structure (I). In some embodiments, the composition comprises from about 65% to about 90% by weight of the compound of structure (I).

Disclosed herein, in certain embodiments, is a composition comprises: a compound of structure (IB) or a mixture of a compound of structure (IB) and its free acid form:

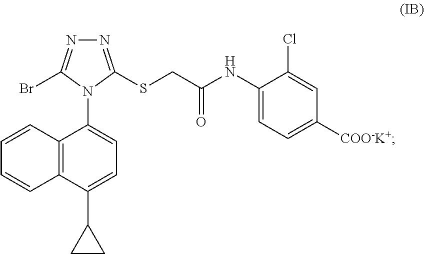

microcrystalline cellulose; hypromellose; methacrylic acid copolymer dispersion; talc; and triethyl citrate.

In some embodiments, the composition comprises: about 214 mg of a compound of structure (IB) or a mixture of a compound of structure (IB) and its free acid form:

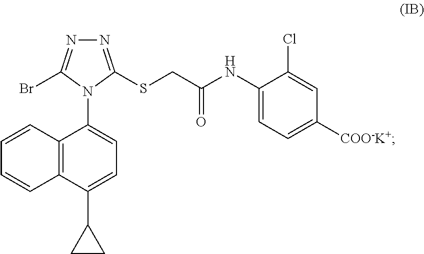

about 35 mg of microcrystalline cellulose; about 29 mg of hypromellose; about 30 mg of methacrylic acid copolymer dispersion; about 6 mg of talc; and about 3 mg of triethyl citrate.

In some embodiments, the composition is a unitary dosage form. In some embodiments, the composition is encapsulated. In some embodiments, the composition is encapsulated within a hard gelatin capsule.

Disclosed herein, in certain embodiments, is a composition comprises: from about 60% to about 90% by weight of a compound of structure (IB) or a mixture of a compound of structure (IB) and its free acid form:

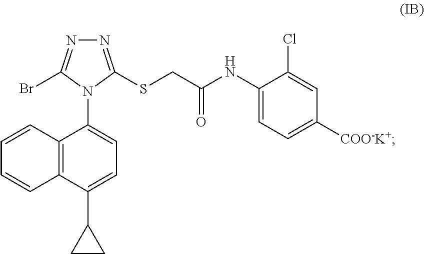

from about 5% to about 15% by weight of microcrystalline cellulose; from about 5% to about 15% by weight of hypromellose; from about 5% to about 15% by weight of methacrylic acid copolymer dispersion; from about 0.5% to about 5% by weight of talc; and from about 0.1% to about 3% by weight of triethyl citrate.

In some embodiments, the composition comprises from about 60% to about 80% by weight of a compound of structure (IB) or a mixture of a compound of structure (IB) and its free acid form. In some embodiments, the composition comprises from about 60% to about 75% by weight of a compound of structure (IB) or a mixture of a compound of structure (IB) and its free acid form. In some embodiments, the composition comprises from about 65% to about 70% by weight of a compound of structure (IB) or a mixture of a compound of structure (IB) and its free acid form. In some embodiments, the composition comprises from about 67% by weight of a compound of structure (IB) or a mixture of a compound of structure (IB) and its free acid form. In some embodiments, the composition comprises from about 7% to about 13% by weight of microcrystalline cellulose. In some embodiments, the composition comprises about 11% by weight of microcrystalline cellulose. In some embodiments, the composition comprises from about 7% to about 11% by weight of hypromellose. In some embodiments, the composition comprises about 9% by weight of hypromellose. In some embodiments, the composition comprises from about 5% to about 15% by weight of o methacrylic acid copolymer dispersion. In some embodiments, the composition comprises from about 8% to about 12% by weight of methacrylic acid copolymer dispersion. In some embodiments, the composition comprises about 10% by weight of methacrylic acid copolymer dispersion. In some embodiments, the composition comprises from about 1% to about 4% by weight of talc. In some embodiments, the composition comprises about 2% by weight of talc. In some embodiments, the composition comprises from about 0.2% to about 2.5% by weight of triethyl citrate. In some embodiments, the composition comprises from about 0.5% to about 2% by weight of triethyl citrate. In some embodiments, the composition comprises about 1% by weight of triethyl citrate. In some embodiments, the composition comprises: about 67% by weight of a compound of structure (IB) or a mixture of a compound of structure (IB) and its free acid form; about 11% by weight of microcrystalline cellulose; about 9% by weight of hypromellose; about 10% by weight of methacrylic acid copolymer dispersion; about 2% by weight of talc; and about 1% by weight of triethyl citrate.

Disclosed herein, in certain embodiments, is a composition comprises: a compound of structure (IB) or a mixture of a compound of structure (IB) and its free acid form:

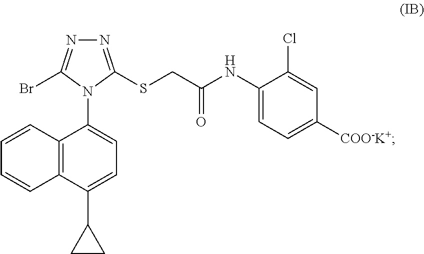

microcrystalline cellulose; and hypromellose; wherein the composition is in the form of granules.

In some embodiments, the granules will not pass through a 40 mesh screen. In some embodiments, the granules are coated with hypromellose. In some embodiments, the coated granules are further coated with a composition comprises: methacrylic acid copolymer dispersion; talc; and triethyl citrate.

In some embodiments, the composition is a unitary dosage form. In some embodiments, the composition is encapsulated. In some embodiments, the composition is encapsulated within a hard gelatin capsule.

Disclosed herein, in certain embodiments, is a composition comprises: about 214 mg of a compound of structure (IB) or a mixture of a compound of structure (IB) and its free acid form:

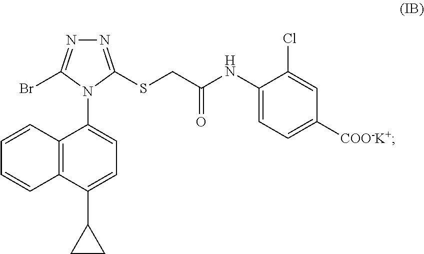

about 35 mg of microcrystalline cellulose; about 13.5 mg of hypromellose; wherein the composition is in the form of granules which do not pass through a 40 mesh screen; and wherein the granules are coated with about 15.3 mg hypromellose; and wherein the coated granules are further coated with a composition comprises: about 30.4 mg of methacrylic acid copolymer dispersion; about 6.1 mg of talc; and about 3.0 mg of triethyl citrate.

In some embodiments, the composition is encapsulated. In some embodiments, the composition is encapsulated within a hard gelatin capsule. In some embodiments, not less than about 85% of the compound of structure (IB) or a mixture of a compound of structure (IB) and its free acid form is released within 30 mins; and not less than about 90% of the compound of structure (IB) or a mixture of a compound of structure (IB) and its free acid form is released within 45 mins; as measured using United States Pharmacopoeia <711> method A, using Apparatus 2 operating at 50 rpm in 900 mL of dissolution medium at pH 6.8 at 37° C.

Disclosed herein, in certain embodiments, is a composition comprises: a compound of structure (I):

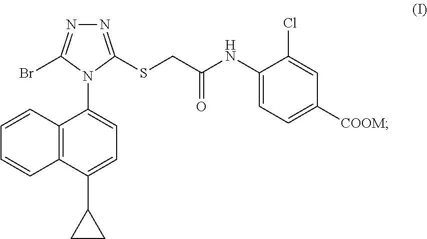

wherein M is hydrogen, sodium, potassium, calcium, or arginine; a diluent; and a disintegrant.

In some embodiments, the diluent is microcrystalline cellulose. In some embodiments, the microcrystalline cellulose comprises from about 40% to about 60% by weight of the composition. In some embodiments, the microcrystalline cellulose comprises about 50% by weight of the composition. In some embodiments, the disintegrant is croscarmellose sodium. In some embodiments, the croscarmellose sodium comprises from about 0.1% to about 2% by weight of the composition. In some embodiments, the croscarmellose sodium comprises from about 0.5% by weight of the composition. In some embodiments, the composition comprises about 50% by weight microcrystalline cellulose; and about 0.5% by weight croscarmellose sodium.

In some embodiments, M is hydrogen or potassium. In some embodiments, M is potassium. In some embodiments, the composition comprises at least 50% by weight of a compound or mixture of compounds of structure (I). In some embodiments, the composition comprises at least 60% by weight of a compound or mixture of compounds of structure (I). In some embodiments, the composition comprises at least 70% by weight of a compound or mixture of compounds of structure (I). In some embodiments, the composition comprises at least 80% by weight of a compound or mixture of compounds of structure (I). In some embodiments, the composition comprises: about 50% by weight of a compound or mixture of compounds of structure (I); about 49.4% by weight of microcrystalline cellulose; and about 0.55% by weight of croscarmellose sodium.

In some embodiments, the composition is a unitary dosage form. In some embodiments, the composition is encapsulated within a capsule. In some embodiments, the capsule comprises hard gelatin. In some embodiments, the hard gelatin capsule is over-encapsulated. In some embodiments, the hard gelatin capsule is over-encapsulated within a hypromellose capsule.

Disclosed herein, in certain embodiments, is a composition comprises: about 100 mg of a compound of structure (IA):

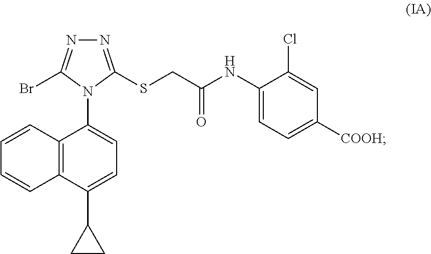

about 98.9 mg of microcrystalline cellulose; and about 1.1 mg of croscarmellose sodium.

In some embodiments, the composition is encapsulated.

Disclosed herein, in certain embodiments, is a composition comprises: about 106.8 mg of a compound of structure (IB) or a mixture of a compound of structure (IB) and its free acid form:

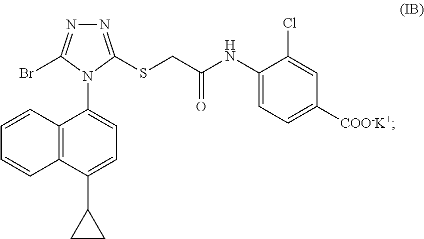

about 92.1 mg of microcrystalline cellulose; and about 1.1 mg of croscarmellose sodium.

Disclosed herein, in certain embodiments, is a composition comprises: about 213 mg of a compound of structure (IB) or a mixture of a compound of structure (IB) and its free acid form:

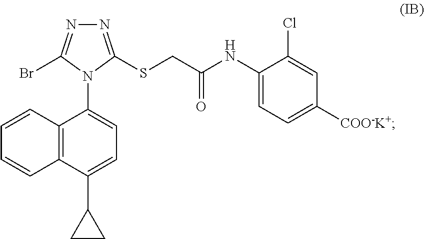

microcrystalline cellulose; and croscarmellose sodium.

Disclosed herein, in certain embodiments, is a composition comprises: about 430 mg of a compound of structure (IB) or a mixture of a compound of structure (IB) and its free acid form:

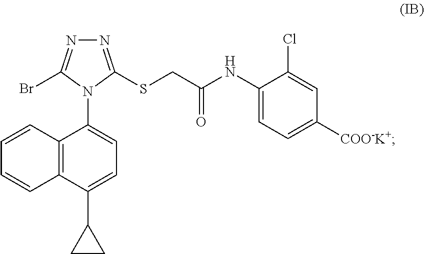

microcrystalline cellulose; and croscarmellose sodium.

Disclosed herein, in certain embodiments, is a composition comprises: about 860 mg of a compound of structure (IB) or a mixture of a compound of structure (IB) and its free acid form:

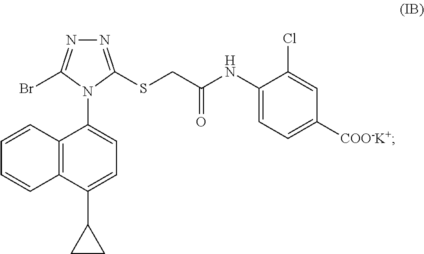

microcrystalline cellulose; and croscarmellose sodium.

Disclosed herein, in certain embodiments, is a composition comprises: about 1000 mg of a compound of structure (IB) or a mixture of a compound of structure (IB) and its free acid form:

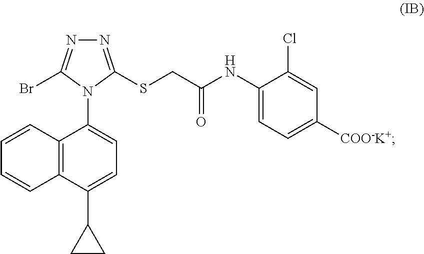

microcrystalline cellulose; and croscarmellose sodium.

In some embodiments, the composition is encapsulated. In some embodiments, the composition is encapsulated within a hard gelatin capsule. In some embodiments, the hard gelatin capsule is over-encapsulated. In some embodiments, the hard gelatin capsule is over-encapsulated within a hypromellose capsule. In some embodiments, not less than about 80% of the compound of structure (IB) dissolves within 30 minutes, as measured using United States Pharmacopoeia Apparatus 1 operating at 75 rpm in 900 mL water at 37° C. In some embodiments, the in vitro dissolution rate as measured using United States Pharmacopoeia Apparatus 1 operating at 75 rpm in 900 mL water at 37° C. is not less than about 80% of the compound of structure (IB) is released within 30 minutes. In some embodiments, not less than about 78% of the compound of structure (IB) dissolves within 30 minutes; and not less than about 95% of the compound of structure (IB) dissolves within 45 minutes; as measured using United States Pharmacopoeia Apparatus 1 operating at 75 rpm in 900 mL water at 37° C.

Disclosed herein, in certain embodiments, is a composition comprises: a compound of structure (I):

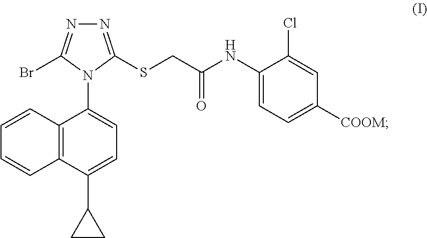

wherein M is hydrogen, sodium, potassium, calcium, or arginine; a diluent; a binder; a lubricant; and a coating.

In some embodiments, the composition is a unitary dosage form. In some embodiments, the composition is a monolithic solid.

Disclosed herein, in certain embodiments, is a composition comprises: about 200 mg of a compound or mixture of compounds of structure (IA):

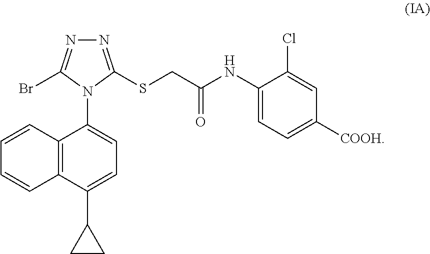

Disclosed herein, in certain embodiments, is a composition comprises: from about 60% to about 90% by weight of a compound or mixture of compounds of structure (IB):

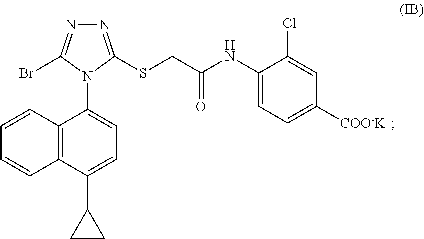

from about 5% to about 15% by weight of microcrystalline cellulose; from about 2.5% to about 10% by weight of acryl-eze white from about 2.5% to about 10% by weight of hypromellose; from about 0.25% to about 2% by weight of magnesium stearate; Disclosed herein, in certain embodiments, is a composition comprises: about 213.6 mg of a compound or mixture of compounds of structure (IB):

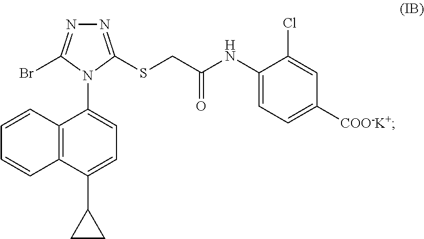

about 34.0 mg of microcrystalline cellulose; about 23.1 mg of hypromellose; about 1.3 mg of magnesium stearate; and about 27.1 mg of acryl-eze white.

In some embodiments, the granules are blended with magnesium stearate. In some embodiments, the composition is compressed into a monolithic solid. In some embodiments, the composition is coated with hypromellose. In some embodiments, the composition is further coated with acryl-eze white. In some embodiments, the in vitro dissolution rate as measured using United States Pharmacopoeia method A, using Apparatus 2 operating at 50 rpm in 700 mL of dissolution medium for two hours at pH 1.2 at 37° C. is about 0% to about 5% of the compound of structure (IB) released within 2 hours; and after 2 hours, in 900 mL of buffer at pH 6.8 at 37° C. is about 15% to about 45% of the compound of structure (IB) released within 30 mins; is about 50% to about 85% of the compound of structure (IB) released within 45 mins; is not less than about 80% of the compound of structure (IB) released within 60 mins; is not less than about 90% of the compound of structure (IB) released within 90 mins; and is not less than about 95% of the compound of structure (IB) released within 100 mins.

Disclosed herein, in certain embodiments, is a method for treating or preventing HIV infection, comprises administering to a subject in need thereof an effective amount of a composition disclosed herein.

Disclosed herein, in certain embodiments, is a method for preventing infection with an immunodeficiency virus in a subject, treating infection with an immunodeficiency virus in a subject, preventing acquired immunodeficiency syndrome (AIDS) in a subject, treating acquired immunodeficiency syndrome (AIDS) in a subject, preventing an AIDS-related complex (ARC) in a subject or treating an AIDS-related complex (ARC) in a subject, comprises administering to the subject an effective amount of a composition disclosed herein.

Disclosed herein, in certain embodiments, is a method of inhibiting an immunodeficiency virus comprises contacting said immunodeficiency virus with a composition disclosed herein.

In some embodiments, the AUCτ (μg·hr/mL) of a composition disclosed herein is between about 0.6 and about 18. In some embodiments, the AUCτ (μg·hr/mL) of a composition disclosed herein is between about 2.5 and about 11. In some embodiments, the AUCτ (μg·hr/mL) of a composition disclosed herein is between about 4.8 and about 8. In some embodiments, the Cmax (μg/mL) of a composition disclosed herein is between about 0.15 and about 6. In some embodiments, the Cmax (μg/mL) of a composition disclosed herein is between about 0.5 and about 5. In some embodiments, the Cmax (μg/mL) of a composition disclosed herein is between about 1 and about 4.

In some embodiments, a composition disclosed herein provides the metabolite M6 (2-(5-bromo-4-(4-cyclopropylnaphthalen-1-yl)-4H-1,2,4-triazol-3-ylthio)acetic acid). In some embodiments, a composition disclosed herein provides an AUCτ (m·hr/mL) of metabolite M6 between about 2.5 and about 30. In some embodiments, a composition disclosed herein provides an AUCτ (μm·hr/mL) of metabolite M6 between about 8 and about 25. In some embodiments, a composition disclosed herein provides an AUCτ (μm·hr/mL) of metabolite M6 between about 12 and about 20. In some embodiments, the Cmax (μg/mL) of metabolite M6 is between about 0.25 and about 4. In some embodiments, the Cmax (μg/mL) of metabolite M6 is between about 1 and about 3. In some embodiments, the Cmax (μg/mL) of metabolite M6 is between about 2.4 and about 3. In some embodiments, the molar ratio of the AUCτ (μm·hr/mL) of the metabolite M6 to the AUCτ (μm·hr/mL) of Compound I is about 2 to about 11. In some embodiments, the molar ratio of the AUCτ (μm·hr/mL) of the metabolite M6 to the AUCτ (μg·hr/mL) of Compound I is about 4 to about 8. In some embodiments, the molar ratio of the Cmax (μg/mL) of the metabolite M6 to the Cmax (μg/mL) of Compound I is about is about 1 to about 7. In some embodiments, the molar ratio of the Cmax (μg/mL) of the metabolite M6 to the Cmax (μg/mL) of Compound I is about is about 2 to about 6.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a flow chart depicting the steps for the preparation of the composition described in example 2A.

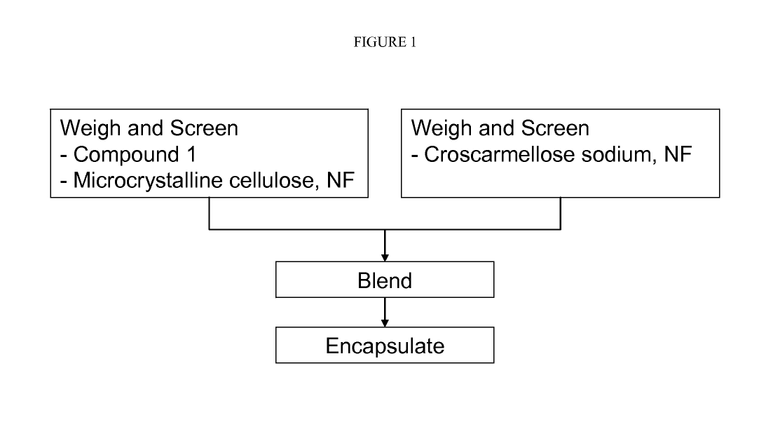

FIG. 2 shows a flow chart depicting the steps for the preparation of the composition described in example 3A.

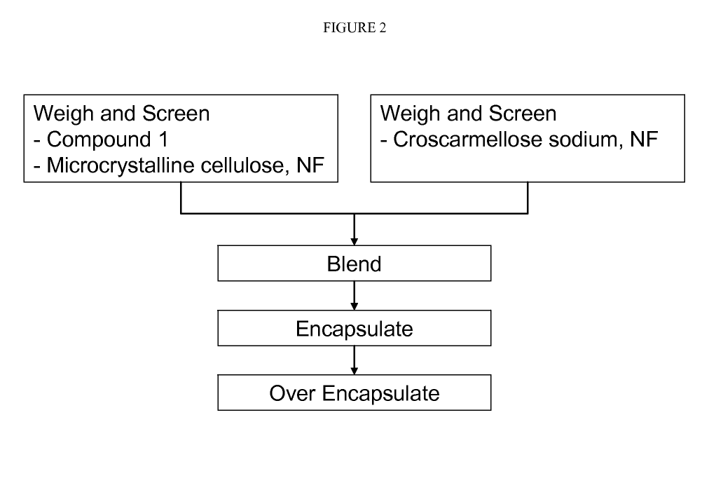

FIG. 3 shows a flow chart depicting the steps for the preparation of the composition described in example 4A.

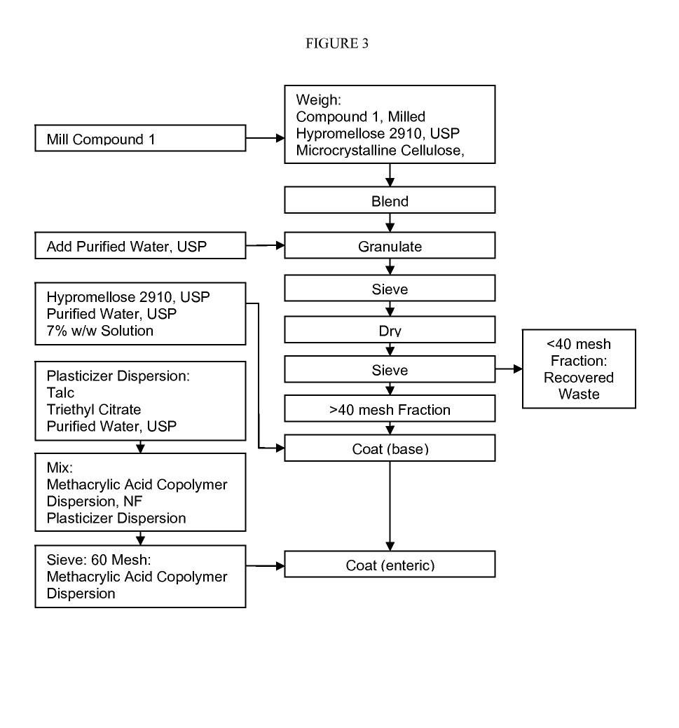

FIG. 4 shows a flow chart depicting the steps for the preparation of the composition described in example 5A.

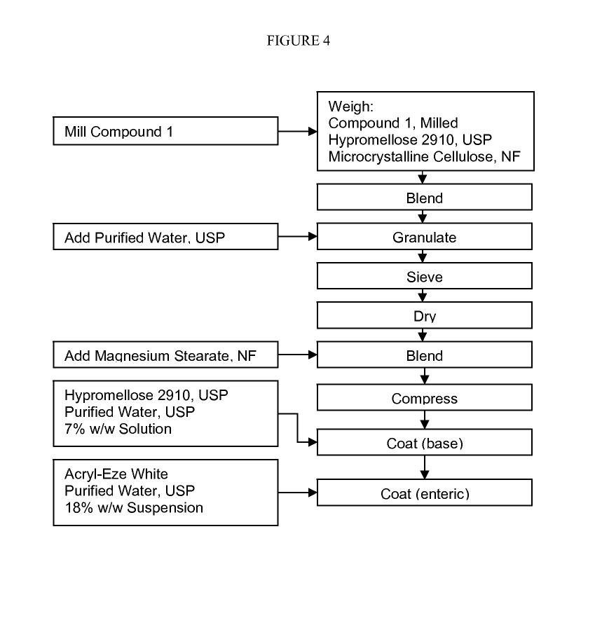

FIG. 5 shows the median change in viral load after administration of compositions comprising compound 1.

FIG. 6 shows viral load reduction and C_{trough} by cohort after administration of compositions comprising compound 1.

FIG. 7 shows CYP3A4 Induction as measured by Fold change in Beta-hydroxycortisol/cortisol after administration of compositions comprising compound 1.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. The section headings used herein are for organizational purposes only and are not to be construed as limiting the subject matter described.

### Pharmaceutical Compositions

Described herein are pharmaceutical compositions comprising 4-(2-(5-bromo-4-(1-cyclopropylnaphthalen-4-yl)-4H-1,2,4-triazol-3-ylthio)acetamido)-3-chlorobenzoic acid or a pharmaceutically acceptable salt thereof. In some embodiments, the pharmaceutical compositions comprise an effective amount of 4-(2-(5-bromo-4-(1-cyclopropylnaphthalen-4-yl)-4H-1,2,4-triazol-3-ylthio)acetamido)-3-chlorobenzoic acid or a pharmaceutically acceptable salt thereof. In some embodiments, the pharmaceutical compositions comprise an effective amount of 4-(2-(5-bromo-4-(1-cyclopropylnaphthalen-4-yl)-4H-1,2,4-triazol-3-ylthio)acetamido)-3-chlorobenzoic acid or a pharmaceutically acceptable salt thereof and at least one pharmaceutically acceptable carrier. In some embodiments the pharmaceutical compositions are for the treatment of disorders. In some embodiments the pharmaceutical compositions are for the treatment of disorders in a mammal. In some embodiments the pharmaceutical compositions are for the treatment of disorders in a human.

### Methods: Viral Infection

The present invention also provides methods useful for preventing or treating viral infection in a subject. The method includes administering an effective amount of a composition as described herein to a subject such as to prevent or treat the viral infection. In some embodiments, the method is for preventing or treating HIV infection, and includes administering to a subject in need thereof an effective amount of a composition as described herein. In further or additional embodiments, the method is for preventing infection with an immunodeficiency virus in a subject, treating infection with an immunodeficiency virus in a subject, preventing acquired immunodeficiency syndrome (AIDS) in a subject, treating acquired immunodeficiency syndrome (AIDS) in a subject, preventing an AIDS-related complex (ARC) in a subject or treating an AIDS-related complex (ARC) in a subject, comprising administering to the subject an effective amount of a composition as described herein.

### Terminology

The term “subject”, “patient” or “individual” as used herein in reference to individuals suffering from a disorder, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “effective amount”, “therapeutically effective amount” or “pharmaceutically effective amount” as used herein, refer to an amount of at least one agent or compound being administered that is sufficient to treat or prevent the particular disease or condition. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

The term “pharmaceutically acceptable” as used herein, refers to a material, such as a carrier or diluent, which does not abrogate the biological activity or properties of the compounds described herein, and is relatively nontoxic, i.e., the material may be administered to an individual without causing undesirable biological effects or interacting in a deleterious manner with any of the components of the composition in which it is contained.

The terms “composition” and “pharmaceutical composition,” as used herein, refer to 4-(2-(5-bromo-4-(1-cyclopropylnaphthalen-4-yl)-4H-1,2,4-triazol-3-ylthio)acetamido)-3-chlorobenzoic acid or a pharmaceutically acceptable salt thereof, optionally mixed with at least one pharmaceutically acceptable chemical component, such as, though not limited to carriers, stabilizers, diluents, dispersing agents, suspending agents, thickening agents, excipients and the like.

The term “pharmaceutically acceptable salt” as used herein, includes salts of 4-(2-(5-bromo-4-(4-cyclopropylnaphthalen-1-yl)-4H-1,2,4-triazol-3-ylthio)acetamido)-3-chlorobenzoate with any pharmaceutically acceptable cation, that retain the biological effectiveness of the free acid. For example, the free acid group of 4-(2-(5-bromo-4-(1-cyclopropylnaphthalen-4-yl)-4H-1,2,4-triazol-3-ylthio)acetamido)-3-chlorobenzoic acid may react with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, N⁻(C₁₋₄ alkyl)₄, and the like. Representative organic amines useful for the formation of base addition salts include arginine, lysine, ethylamine, diethylamine, ethylenediamine, ethanolamine, diethanolamine, piperazine and the like. Salts can be prepared in situ during final isolation and purification, or by separate reaction and isolation of the salt thus formed.

### Doses

The total amount of 4-(2-(5-bromo-4-(1-cyclopropylnaphthalen-4-yl)-4H-1,2,4-triazol-3-ylthio)acetamido)-3-chlorobenzoic acid or pharmaceutically acceptable salt thereof, administered will firstly be dependent on the mammal being treated. In the instances where administration is to a human subject, the amount will normally be determined by the prescribing physician with the dosage generally varying according to the age, sex, diet, weight, general health and response of the individual patient, the severity of the patient's symptoms, the precise indication or condition being treated, the severity of the indication or condition being treated, time of administration, route of administration, the disposition of the composition, rate of excretion, drug combination, and the discretion of the prescribing physician. The pharmaceutical composition may be in unit dosage form. In such form, the preparation is subdivided into unit doses containing appropriate quantities of the active component, e.g., an effective amount to achieve the desired purpose. Determination of the proper dosage for a particular situation is within the skill of the art. For convenience, the total daily dosage may be divided and administered in portions during the day if desired. The amount and frequency of administration, and if applicable other therapeutic agents and/or therapies, will be regulated according to the judgment of the attending clinician (physician) considering such factors as described above. Thus the amount of pharmaceutical composition to be administered may vary widely. Administration may occur in an amount of between about 0.001 mg/kg of body weight to about 100 mg/kg of body weight per day (administered in single or divided doses), or at least about 0.1 mg/kg of body weight per day. A particular therapeutic dosage includes, e.g., from about 0.01 mg to about 7000 mg of compound, or, e.g., from about 0.05 mg to about 2500 mg. The quantity of 4-(2-(5-bromo-4-(1-cyclopropylnaphthalen-4-yl)-4H-1,2,4-triazol-3-ylthio)acetamido)-3-chlorobenzoic acid, or a pharmaceutically acceptable salt thereof in a unit dose may occur in an amount of between about 1 mg to 3000 mg, from about 2 mg to 2000 mg, or 10 mg to 2000 mg, according to the particular application. In some embodiments, the amount is from about 100 mg to about 1500 mg, from about 150 mg to about 1200 mg, or from about 200 mg to about 1000 mg. In further or additional embodiments, the amount is at least 100 mg, at least 200 mg, at least 250 mg, at least 300 mg, at least 400 mg, at least 500 mg, at least 600 mg, at least 700 mg, at least 750 mg, at least 800 mg, at least 900 mg or at least 1000 mg. In further or additional embodiments, the amount is about 100 mg, about 200 mg, about 250 mg, about 300 mg, about 400 mg, about 500 mg, about 600 mg, about 700 mg, about 750 mg, about 800 mg, about 900 mg or about 1000 mg. In some embodiments the compositions are administered once daily. In further or additional embodiments, the compositions are administered twice daily. In further or additional embodiments, the compositions are administered at least twice daily. In some instances, dosage levels below the lower limit of the aforesaid range may be more than adequate, while in other cases still larger doses may be employed without causing any harmful side effect, e.g. by dividing such larger doses into several small doses for administration throughout the day. In combinational applications in which the compound is not the sole therapy, it may be possible to administer lesser amounts of compound and still have therapeutic or prophylactic effect.

### Dosage Forms

The pharmaceutical compositions described herein are typically useful for oral administration as solid dosage forms, such as tablets, capsules, pills, powders, granules and the like. They may be administered for immediate release, delayed release or sustained release of 4-(2-(5-bromo-4-(1-cyclopropylnaphthalen-4-yl)-4H-1,2,4-triazol-3-ylthio)acetamido)-3-chlorobenzoic acid, or a pharmaceutically acceptable salt thereof.

### Drug Combinations

The pharmaceutical compositions described herein may further comprise other drugs. In some embodiments the other drugs may be anti-viral, anti-HIV or anti-AIDS drugs. In further or additional embodiments the other drugs include but are not limited to Abacavir, Abacavir sulfate, Amprenavir, Atazanavir, Atazanavir sulfate, Darunavir, Didanosine, Delaviridine, Enteric coated didanosine, Efavirenz, Emtricitabine, Enfuvirtide, Etravirine, Fasamprenavir calcium, Indinavir, Lamivudine, Lopinavir, Maraviroc, Nelfinavir, Nelfinavir mesylate, Nevirapine, Raltegravir, Ritonavir, Saquinavir, Saquinavir mesylate, Stavudine, Tenofovir, Tenofovir disoproxil fumarate, Tipranavir, Zalcitabine, Zidovudine, ATRIPLA™, COMBIVIR™, EMTRIVA™, EPIVIR™, EPZICOM™, HIVID™, RETROVIR™, TRIZIVIR™, TRUVADA™, VIDEX EC™, VIDEX™, VIREAD™, ZERIT™, ZIAGEN™, INTELENCE™, RESCRIPTOR™, SUSTIVA™, VIRAMUNE™, AGENERASE™, APTIVUS™, CRIXIVAN™, INVIRASE™, KALETRA™, LEXIVA™, NORVIR™, PREZISTA™, REYATAZ™, VIRACEPT™, FUZEON™, SELZENTRY™, ISENTRESS™, or a combination thereof. In those embodiments wherein the pharmaceutical compositions further comprise other drugs, the amount of 4-(2-(5-bromo-4-(1-cyclopropylnaphthalen-4-yl)-4H-1,2,4-triazol-3-ylthio)acetamido)-3-chlorobenzoic acid included may be less than or the same as those compositions wherein 4-(2-(5-bromo-4-(1-cyclopropylnaphthalen-4-yl)-4H-1,2,4-triazol-3-ylthio)acetamido)-3-chlorobenzoic acid is the sole active component. In some embodiments the amount of 4-(2-(5-bromo-4-(1-cyclopropylnaphthalen-4-yl)-4H-1,2,4-triazol-3-ylthio)acetamido)-3-chlorobenzoic acid may be less than 50% by weight of the total composition.

### Kits

The compositions and methods described herein provide kits for the treatment of diseases and disorders. These kits comprise a compositions described herein in a container and, optionally, instructions teaching the use of the kit. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

The compositions described herein are also useful for diagnostics and as research reagents. For example, the compounds described herein, either alone or in combination with other compounds, may be useful as tools in differential and/or combinatorial analyses to elucidate expression patterns of genes expressed within cells and tissues. As one non-limiting example, expression patterns within cells or tissues treated with one or more compounds are compared to control cells or tissues not treated with compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Besides being useful for human treatment, the compositions described herein may also be useful for veterinary treatment of other animals.

### EXAMPLES

The examples and preparations provided below further illustrate and exemplify the compounds of the present invention and methods of preparing such compounds. It is to be understood that the scope of the present invention is not limited in any way by the scope of the following examples and preparations.

### I Chemical Syntheses

### Example 1

### Synthesis of 4-(2-(5-bromo-4-(1-cyclopropylnaphthalen-4-yl)-4H-1,2,4-triazol-3-ylthio)acetamido)-3-chlorobenzoic acid (compound 1)

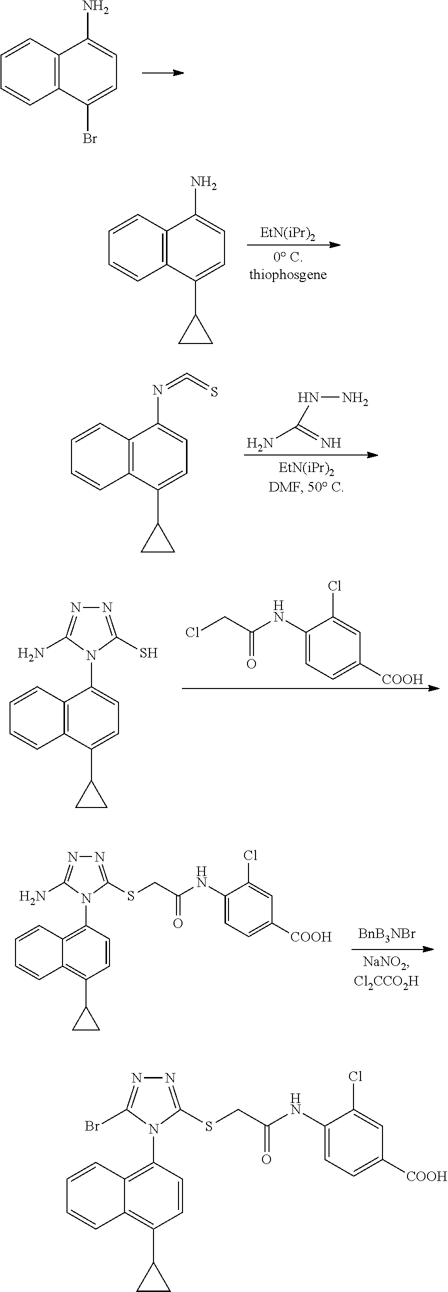

4-(2-(5-Bromo-4-(1-cyclopropylnaphthalen-4-yl)-4H-1,2,4-triazol-3-ylthio)acetamido)-3-chlorobenzoic acid (compound 1) was prepared as described previously (see US published patent application US 2006-0270725) and outlined below.

### 4-Cyclopropylnaphthalen-1-amine

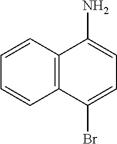

Palladium acetate (23 mg, 0.1 mmol) was added to a solution of 4-bromonaphthalen-1-amine (500 mg, 2.01 mmol), cyclopropyl boronic acid (225 mg, 2.62 mmol), potassium phosphate (1.49 g, 7.04 mmol) and tricyclohexylphosphine (56 mg, 0.2 mmol) in toluene (10 mL) and water (0.4 mL) under nitrogen atmosphere. The mixture was heated at 100° C. for 3 h and then cooled to room temperature. Water was added and the mixture extracted with ethyl acetate, dried over sodium sulfate and concentrated to give crude 4-cyclopropylnaphthalen-1-amine (550 mg) which was used in the next step without further purification.

### 1-Cyclopropyl-4-isothiocyanatonaphthalene

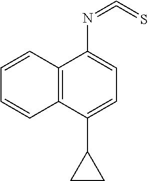

Sodium bicarbonate (7 mL, sat. solution) and thiophosgene (0.2 mL, 2.6 mmol) were added to a solution of 4-cyclopropylnaphthalen-1-amine (440 mg, 2.6 mmol) in dichloromethane (14 mL) and the mixture stirred at room temperature for 1 h. The organic layer was separated, dried over sodium sulfate and concentrated to give crude 1-cyclopropyl-4-isothiocyanatonaphthalene (877 mg, 99%) which was used in the next step without further purification.

### 5-Amino-4-(1-cyclopropylnaphthalen-4-yl)-4H-1,2,4-triazole-3-thiol

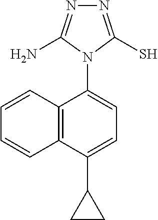

Aminoguanidine hydrochloride (355 mg, 3.2 mmol) and diisopropyl ethylamine (0.56 mL, 3.2 mmol) were added to a solution of 1-cyclopropyl-4-isothiocyanatonaphthalene (447 mg, 2.1 mmol) in dimethylformamide (DMF, 3 mL) and the mixture stirred at 50° C. for 18 hours. The mixture was then concentrated and aqueous sodium hydroxide solution (2M, 10 mL) added. The mixture was stirred at 50° C. for 18 hours, cooled to room temperature and neutralized with aqueous 1N HCl. The resulting precipitate was isolated to give 5-amino-4-(1-cyclopropylnaphthalen-4-yl)-4H-1,2,4-triazole-3-thiol (240 mg, 44%).

### 4-(2-(5-Amino-4-(1-cyclopropylnaphthalen-4-yl)-4H-1,2,4-triazol-3-ylthio)acetamido)-3-chlorobenzoic acid

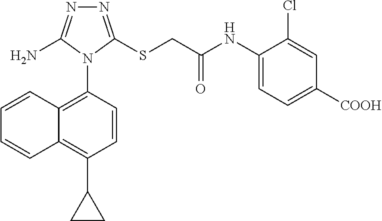

5-Amino-4-(1-cyclopropylnaphthalen-4-yl)-4H-1,2,4-triazole-3-thiol (789 mg, 2.79 mmol) and 3-chloro-4-(2-chloroacetamido)benzoic acid (693 mg, 2.79 mmol) were dissolved in DMF (6 mL) and the solution stirred at 50° C. for 18 hours. Water was then added and the mixture extracted with ethyl acetate. The organic layer was separated, dried over sodium sulfate and concentrated to give 4-(2-(5-amino-4-(1-cyclopropylnaphthalen-4-yl)-4H-1,2,4-triazol-3-ylthio)acetamido)-3-chlorobenzoic acid (1.04 g, 75%).

### 4-(2-(5-Bromo-4-(1-cyclopropylnaphthalen-4-yl)-4H-1,2,4-triazol-3-ylthio)acetamido)-3-chlorobenzoic acid

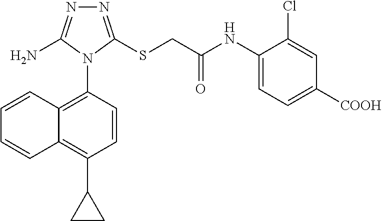

Dichloroacetic acid (0.35 mL, 4.2 mmol) was added to a mixture of give 4-(2-(5-amino-4-(1-cyclopropylnaphthalen-4-yl)-4H-1,2,4-triazol-3-ylthio)acetamido)-3-chlorobenzoic acid (1.04 g, 2.1 mmol), benzyltriethyl ammonium bromide (1.65 g, 6.1 mmol) and sodium nitrite (2.9 g, 42.1 mmol) in dibromomethane (44 mL). The mixture was stirred at room temperature for 18 hours in the dark, then concentrated and the resulting residue purified by column chromatography (95% dichloromethane/5% methanol) to give 4-(2-(5-bromo-4-(1-cyclopropylnaphthalen-4-yl)-4H-1,2,4-triazol-3-ylthio)acetamido)-3-chlorobenzoic acid (393 mg, 34%).

### II Preparation and Analysis of Compositions

### Example 2A

### Preparation of Immediate Release Capsules, 100 mg

4-(2-(5-Bromo-4-(1-cyclopropylnaphthalen-4-yl)-4H-1,2,4-triazol-3-ylthio)acetamido)-3-chlorobenzoic acid (compound 1, prepared as described herein) and microcrystalline cellulose were screened through a #40 mesh screen. The screened mixture and croscarmellose sodium were blended together using a Turbular Type T10B Shaker Mixer for approximately 15 minutes. 200 mg of the powder blend was encapsulated into a size # 2 dark green opaque hard gelatin Coni-Snap capsule.

Two batches of capsules were prepared according to this procedure; the first batch size was 3380 capsules and the second 4400 capsules. The capsules were analyzed for identity, strength, purity, content uniformity and dissolution profiles, as described below.

### Unit Composition

| | Amount |
|---|---|
| Ingredients | (mg/capsule) |
| Compound 1 | 100.0* |
| Microcrystalline cellulose, NF | 98.9 |
| Croscarmellose sodium, NF | 1.1 |
| Total Fill Weight | 200.0 |
| Size 2 dark green opaque Coni-Snap | 1 |
| capsule | |

### Batch Composition

100 mg Capsule

| | | g/3200 |
|---|---|---|
| Ingredients | mg/capsule | capsules |
| Compound 1 | 100.0* | 320.0* |
| Microcrystalline | 98.9 | 316.5 |
| cellulose, NF | | |
| Croscarmellose sodium, | 1.1 | 3.5 |
| NF | | |

*As free acid, equivalent to 106.8 mg of potassium salt. The quantity of compound 1 used was adjusted based on the actual potency and a corresponding adjustment with microcrystalline cellulose made.

### Example 2B

### Identification of Compound 1 in Capsules Prepared in Example 2A

An isocratic reversed-phase HPLC method, (4.6×150 mm YMC ODS AQ, 3 μm size particles column; mobile phase of 45% 10 mM KH₂PO₄ (pH 3.0) and 55% acetonitrile) with UV detection at 220 nm, was used to confirm the identity of compound 1. The capsules were extracted with 50/50 (v/v) acetonitrile/water. The identity of compound 1 was determined by comparing the retention time of the peak in the sample preparation with that of a standard preparation and showed relative retention values (Rr) 0.97-1.03 relative to reference standard, confirming the presence of compound 1.

### Example 2C

### Determination of Quantity of Compound 1 in Capsules Prepared in Example 2A

An isocratic reversed-phase HPLC method, (4.6×150 mm YMC ODS AQ, 3 μm size particles column,; mobile phase of 45% 10 mM KH₂PO₄ (pH 3.0) and 55% acetonitrile) with UV detection at 220 nm, was used to determine the amount of compound 1 present. The capsules were extracted with 50/50 (v/v) acetonitrile/water. The quantity of compound 1 present was determined by comparing sample peaks with standard preparations obtained concomitantly.

### Example 2D

### Determination of Impurities in Capsules Prepared in Example 2A

A gradient elution reversed-phase HPLC method, (4.6×150 mm YMC ODS AQ, 3 μm size particles column; mobile phase A 10 mM KH₂PO₄ (pH 3.0) and mobile phase B acetonitrile) with UV detection at 220 nm, was used to determine impurity content. The capsules were extracted with 50/50 (v/v) acetonitrile/water. Impurities were determined by comparing the impurity peak areas in the sample preparation chromatogram to the area of the compound 1 peak in the standard preparation obtained concomitantly. Known impurities were calculated by applying the response factor of the respective impurities. The method reporting limit was 0.05%. The results of the analyses are shown in the table below.

| Impurity | Limit | Batch 1 | Batch 2 |
|---|---|---|---|
| “bromo acid” | NMT 1.0% | not detected | 0.06% |
| impurity | | | |
| “des-bromo” | NMT 1.0% | 0.3% | 0.41% |
| impurity | | | |
| “methyl ester” | NMT 1.0% | 0.4% | 0.18% |
| impurity | | | |
| “des- | NMT 1.5% | not detected | not detected |
| cyclopropyl” | | | |
| impurity | | | |
| Unspecified | NMT 0.5% each, | RRT 0.60: 0.1% | RRT 0.62: 0.06% |
| substances | report RRT | RRT 0.64: 0.1% | RRT 0.85: 0.07% |
| | | RRT 0.98: 0.1% | RRT 1.06: 0.07% |
| | | RRT 1.14: 0.2% | RRT 1.14: 0.13% |
| Total related | NMT 4.0% | 1.0% | 0.99% |
| substances | | | |

NMT—not more than;
RRT = relative retention time

### Example 2E

### Determination of Water Content in Capsules Prepared in Example 2A

Water content was determined using the USP <921>, Karl Fischer method. The results are shown below:

| | Batch 1 | Batch 2 |
|---|---|---|
| Water content | 4.65% | 5.71% |

### Example 2F

### Dissolution Profile of Compound 1 from Capsules Prepared in Example 2A

Dissolution of compound 1 from the capsules was determined in 900 mL of water at 37° C. using USP Apparatus 1 operating at 75 rpm. A filtered sample of the dissolution medium was taken at the specified time(s) and analyzed by an HPLC procedure using the same chromatographic conditions as for content determination, as described above. The release was determined by comparing the peak responses of the sample chromatograms to the peak responses of the standard chromatograms obtained concomitantly and the results are shown below.

| Limit | Batch 1 | Batch 2 |
|---|---|---|
| NLT 80% | Mean: 96.7% | Mean: 107.4% |
| (Q = 75%) in 30 | RSD: 1.5% | RSD: 3.1% |
| minutes | Min: 94.4% | Min: 103.4% |
| | Max: 98.9% | Max: 111.4% |

NLT = not less than;
RRT = relative retention time

### Example 3A

### Preparation of Over-Encapsulated Capsules, 100 mg

Size 1 white Vcaps capsule shells were loaded into Profill encapsulator trays and the Vcaps caps were separated from the capsule bodies. A size #2 dark green opaque hard gelatin Coni-Snap capsule (prepared as described in example 2A above) was placed into each of the Vcaps capsule bodies. The capsule caps were placed back onto capsule bodies using the Profill equipment and gently pressed shut to secure the caps on the bodies. This process was repeated using additional trays as necessary.

Two batches of capsules were prepared according to this procedure; the first batch size was 680 capsules and the second 2200 capsules. The capsules were analyzed for identity, strength, purity, content uniformity and dissolution profiles, as described below.

### Unit Composition

| | Amount |
|---|---|
| Ingredients | (mg/capsule) |
| Compound 1 | 100.0^{a} |
| Microcrystalline cellulose NF | 98.9 |
| Croscarmellose sodium, NF | 1.1 |
| Total Fill Weight | 200.0 |
| Size 2 dark green opaque Coni-Snap | 1 |
| capsule | |
| (first encapsulation) | |
| Size 1 white Vcaps ®, HPMC ConiSnap | 1 |
| capsule | |
| (over encapsulation) | |

^{a}As free acid, equivalent to 106.8 mg of potassium salt. The quantity of compound 1 used was adjusted based on the actual potency and a corresponding adjustment with microcrystalline cellulose made.

### Example 3B

### Identification of Compound 1 in Capsules Prepared in Example 3A

An isocratic reversed-phase HPLC method, (4.6×150 mm YMC ODS AQ, 3 μm size particles column; mobile phase of 45% 10 mM KH₂PO₄ (pH 3.0) and 55% acetonitrile) with UV detection at 220 nm, was used to confirm the of identity of compound 1. The capsules were extracted with 50/50 (v/v) acetonitrile/water. The identity of compound 1 was determined by comparing the retention time of the peak in the sample preparation with that of a standard preparation and showed relative retention values (Rr) 0.97-1.03 relative to reference standard, confirming the presence of compound 1.

### Example 3C

### Determination of Quantity of Compound 1 in Capsules Prepared in Example 3A

An isocratic reversed-phase HPLC method, (4.6×150 mm YMC ODS AQ, 3 μm size particles column,; mobile phase of 45% 10 mM KH₂PO₄ (pH 3.0) and 55% acetonitrile) with UV detection at 220 nm, was used to determine the amount of compound 1 present. The capsules were extracted with 50/50 (v/v) acetonitrile/water. The quantity of compound 1 present was determined by comparing sample peaks with standard preparations obtained concomitantly and is shown below.

| | Batch 1 | Batch 2 |
|---|---|---|
| Amount present | 101.6% | 103.5% |

### Example 3D

### Determination of Impurities in Capsules Prepared in Example 3A

A gradient elution reversed-phase HPLC method, (4.6×150 mm YMC ODS AQ, 3 μm size particles column; mobile phase A 10 mM KH₂PO₄ (pH 3.0) and mobile phase B acetonitrile) with UV detection at 220 nm, was used to determine impurity content. The capsules were extracted with 50/50 (v/v) acetonitrile/water. Impurities were determined by comparing the impurity peak areas in the sample preparation chromatogram to the area of the compound 1 peak in the standard preparation obtained concomitantly. Known impurities were calculated by applying the response factor of the respective impurities. The method reporting limit was 0.05%. The results of the analyses are shown in the table below.

| | Limit | Batch 1 | Batch 2 |
|---|---|---|---|
| “bromo acid” impurity | NMT 1.0% | 0.05% | 0.06% |
| “des-bromo” impurity | NMT 1.0% | 0.33% | 0.41% |
| “methyl ester” impurity | NMT 1.0% | 0.36% | 0.19% |
| “des-cyclopropyl” | NMT 1.5% | 0.08% | Not detected |
| impurity | | | |
| Unspecified substances | NMT 0.5% | RRT 0.07: | RRT 0.62: |
| | each, | 0.05% | 0.06% |
| | report RRT | RRT 0.63: | RRT 0.85: |
| | | 0.06% | 0.08% |
| | | RRT 0.89: | RRT 1.06: |
| | | 0.05% | 0.07% |
| | | RRT 1.06: | RRT 1.14: |
| | | 0.05% | 0.15% |
| | | RRT 1.14: | |
| | | 0.05% | |
| | | RRT 1.15: | |
| | | 0.14% | |
| Total related substances | NMT 4.0% | 1.21% | 1.01% |

NMT—not more than;
RRT = relative retention time

### Example 3E

### Determination of Water Content in Ccapsules Prepared in Example 3A

Water content was determined using the USP <921>, Karl Fischer method. The results are shown below:

Batch number

| | 1 | 2 |
|---|---|---|
| Water content | 6.14% | 6.40% |

### Example 3F

### Dissolution Profile of Compound 1 from Capsules Prepared in Example 3A

Dissolution of compound 1 from the capsules was determined in 900 mL of water at 37° C. using USP Apparatus 1 operating at 75 rpm. A filtered sample of the dissolution medium was taken at the specified times (15, 30, 45 and 60 minutes) and analyzed by HPLC using the same chromatographic conditions as described above for content determination. Compound release was determined by comparing the peak responses of the sample chromatograms to the peak responses of the standard chromatograms obtained concomitantly and the results are shown below.

| Batch number | 1 | | | | 2 | | | |
|---|---|---|---|---|---|---|---|---|
| Dissolution profile | Ave. | RSD | Min. | Max. | Ave. | RSD | Min. | Max. |
| | 15 min = 7% | 122% | 0% | 20% | 15 min = 8% | 163% | 0% | 34% |
| | 30 min = 92% | 11% | 77% | 102% | 30 min = 93% | 4% | 87% | 97% |
| | 45 min = 101% | 2% | 98% | 103% | 45 min = 107% | 3% | 101% | 109% |
| | 60 min = 101% | 2% | 98% | 103% | 60 min = 107% | 3% | 102% | 109% |

NLT = not less than;
RRT = relative retention time

### Example 4A

### Preparation of Enteric Coated Granules, 200 mg

4-(2-(5-Bromo-4-(1-cyclopropylnaphthalen-4-yl)-4H-1,2,4-triazol-3-ylthio)acetamido)-3-chlorobenzoic acid (compound 1, prepared as described herein) was milled and then blended in a planetary mixer with microcrystalline cellulose and hypromellose. The blended ingredients in the planetary mixer were granulated with purified water and the wet granulation was sieved through a No. 8 mesh screen and dried on trays in an oven at 40° C. to a moisture content of less than 5% as determined by loss on drying (LOD). The dried granulation was sieved through a No. 40 mesh screen; the granules retained on the 40 mesh screen were collected, and the remaining fines transferred to recovered waste. Using a fluidized bed coating unit, a hypromellose solution (7% w/w in purified water) was applied to the >40 mesh granules, followed by application of a talc, triethyl citrate and methacrylic acid dispersion (previously sieved through an 80 mesh screen). Approximately 317 mg of the coated granules was manually filled into each of 1700 hard gelatin capsules, Swedish Orange Opaque, Size 1.

One batch of 1700 capsules was prepared according to this procedure. The capsules were analyzed for identity, strength, purity, content uniformity and dissolution profiles, as described below.

### Unit Composition

| | Amount |
|---|---|
| Ingredients | (mg/capsule) |
| Compound 1, Milled | 200.0^{a} |
| Microcrystalline cellulose, NF | 34.9 |
| Hypromellose 2910, USP | 13.5 |
| Total Weight of Uncoated Granules | 262.0 |
| Hypromellose 2910, USP (Base coat) | 15.3 |
| Methacrylic Acid Copolymer Dispersion, | 30.4 |
| NF (Eudragit L30D-55) (Enteric coat) | |
| Talc, USP | 6.1 |
| Triethyl Citrate, NF | 3.0 |
| Empty Hard Gelatin Capsule, Swedish | 1 ea |
| Orange Opaque, Size 1 (Encapsulation) | |
| Total Capsule Fill Weight | 316.8 |

### Batch Composition

200 mg Capsule

| | | g/1,700 |
|---|---|---|
| Ingredients | mg/Capsule | Capsules |
| Active Ingredient | | |
| Compound 1, Milled | 200.0^{a} | 340.0^{a} |
| Granulation Medium | | |
| Purified Water, USP^{b} | — | q.s.^{b} |
| Excipients | | |
| Microcrystalline cellulose, NF | 34.9 | 59.3 |
| Hypromellose 2910, USP | 13.5 | 23.0 |
| Total Uncoated Granule Weight | 262.0 | 445.4 |
| Aqueous Coatings | | |
| Hypromellose 2910, USP | 15.3 | 26.0 |
| Methacrylic Acid Copolymer | 30.4 | 147.3^{c} |
| Dispersion, NF | | |
| (Eudragit L30D-55)^{c} | | |
| Talc, USP | 6.1 | 10.4 |
| Triethyl Citrate, NF | 3.0 | 5.1 |
| Empty Hard Gelatin Capsules, | 1 ea. | 1700 |
| Swedish Orange Opaque, Size 1 | | |
| Total Capsule Fill Weight | 316.8 | 538.6 |

^{a}As free acid, equivalent to 213.6 mg of potassium salt. The quantity of compound 1 used was adjusted based on the actual potency and a corresponding adjustment with microcrystalline cellulose made.
^{b}Quantity sufficient to facilitate granule formation, removed during the drying process
^{c}Eudragit L30D-55 is a dispersion containing 30% solids

### Example 4B

### Identification of Compound 1 in Capsules Prepared in Example 4A

An isocratic reversed-phase HPLC method, (4.6×150 mm YMC ODS AQ, 3 μm size particles column; mobile phase of 45% 10 mM KH₂PO₄ (pH 3.0) and 55% acetonitrile) with UV detection at 220 nm, was used to confirm the identity of compound 1. The capsules were extracted with 20:80 (v/v) methanol/phosphate buffer (pH 7.4) and diluted 1:10 with methanol/water (20:80 v/v ratio). The target concentration of compound 1 for assay is 0.1 mg/mL. The identity of compound 1 was determined by comparing the retention time of the peak in the sample preparation with that of a standard preparation and showed relative retention values (Rr) 1.00±0.03 relative to reference standard, confirming the presence of compound 1.

### Example 4C

### Determination of Quantity of Compound 1 in Capsules Prepared in Example 4A

An isocratic reversed-phase HPLC method, (4.6×150 mm YMC ODS AQ, 3 μm size particles column; mobile phase of 45% 10 mM KH₂PO₄ (pH 3.0) and 55% acetonitrile) with UV detection at 220 nm, was used to determine the amount of compound 1 present. The capsules were extracted with extracted with 20:80 (v/v) methanol/phosphate buffer (pH 7.4) and diluted 1:10 with methanol/water (20:80 v/v ratio). The target concentration of compound 1 for assay is 0.1 mg/mL. The quantity of compound 1 present was determined by comparing sample peaks with standard preparations obtained concomitantly, and confirmed the quantity to be 98.2%, within the 90.0-110.0% limit set.

### Example 4D

### Determination of Impurities in Capsules Prepared in Example 4A

A gradient elution reversed-phase HPLC method, (4.6×150 mm YMC ODS AQ, 3 μm size particles column; mobile phase A 10 mM KH₂PO₄ (pH 3.0) and mobile phase B acetonitrile) with UV detection at 220 nm, was used to determine impurity content. The capsules were extracted with 20/80 (v/v) methanol/phosphate buffer (pH 7.4). Impurities were determined by comparing the impurity peak areas in the sample preparation chromatogram to the area of the compound 1 peak in the standard preparation obtained concomitantly. Known impurities were calculated by applying the response factor of the respective impurities. The method reporting limit was 0.05%. The results of the analyses are shown in the table below.

| | Limit | Results |
|---|---|---|
| “bromo acid” impurity | NMT 1.0% | 0.07%, |
| “des-bromo” impurity | NMT 1.0% | 0.39% |
| “methyl ester” | NMT 1.0% | 0.20% |
| impurity | | |
| “des-cyclopropyl” | NMT 1.5% | ND |
| impurity | | |
| Unspecified | NMT 0.5% each, | RRT 0.63 = 0.06% |
| substances | report RRT | RRT 0.66 = 0.07% |
| | | RRT 0.86 = 0.05% |
| | | RRT 1.02 = 0.07% |
| | | RRT 1.06 = 0.06% |
| | | RRT 1.13 = 0.07% |
| | | RRT 1.22 = 0.06% |
| Total related | NMT 4.0% | 1.10% |
| substances | | |

### Example 4E

### Determination of Water Content in Capsules Prepared in Example 4A

Water content was determined using the USP <921>, Karl Fischer method and measured at 6.9%.

### Example 4F

### Dissolution Profile of Compound 1 from Capsules Prepared in Example 4A

Dissolution of compound 1 from the capsules was determined following the principles of the USP <711> method A for delayed-release dosage forms, using USP Apparatus 2 at 37° C., set at 50 rpm. Acid stage was performed for 2 hours in 700 mL of dissolution medium at pH 1.2 followed by buffer stage performed in 900 mL of dissolution medium at pH 6.8. A filtered aliquot of the test dissolution medium was taken at the specified times (15, 30, 45 and 60 minutes) and analyzed by HPLC using the same chromatographic conditions as described above for content determination. Compound release was determined by comparing the peak responses of the sample chromatograms to the peak responses of the standard chromatograms obtained concomitantly and the results are shown below.

| Limit | Results | |
|---|---|---|
| Acid Stage | Mean = 0.0% | |
| Level 1: NMT 10% for avg | % RSD = 82.0 | |
| of 6 units and no individual | Min = 0.0% | |
| greater than 25% dissolved | Max = 0.0%, Pass | |
| Buffer Stage | 30 min | Mean = 93.0% |
| Report profile at t = 30, 45, | | % RSD = 2.9% |
| 60, 90, and 100 min | | Min = 88.5% |
| | | Max = 95.9% |
| | 45 min | Mean = 95.9% |
| | | % RSD = 2.6% |
| | | Min = 91.3% |
| | | Max = 98.2% |
| | 60 min | Mean = 96.8% |
| | | % RSD = 2.7% |
| | | Min = 91.8% |
| | | Max = 99.2% |
| | 90 min | Mean = 97.6% |
| | | % RSD = 3.1% |
| | | Min = 92.0% |
| | | Max = 100.2% |
| | 100 min | Mean = 98.4% |
| | | % RSD = 3.8% |
| | | Min = 91.9% |
| | | Max = 101.6% |

NMT = not more than;
RRT = relative retention time

### Example 5A

### Preparation of Enteric Coated Tablets, 200 mg

4-(2-(5-Bromo-4-(1-cyclopropylnaphthalen-4-yl)-4H-1,2,4-triazol-3-ylthio)acetamido)-3-chlorobenzoic acid (compound 1, prepared as described herein) was milled through a Fitzmill fitted with a 0.033″ round-hole screen. The milled compound 1, microcrystalline cellulose and hypromellose were blended with a Robot Coupe RSI-3VG high-shear roto-granulator for approximately 5 minutes and the blended ingredients granulated with purified water. The wet granulation was sieved through a No. 10 mesh screen and dried on trays in an oven at 40° C. to a moisture content of NMT 3% as determined by loss on drying (LOD). The dried granulation was sieved through a No. 10 mesh screen and approximately half of the granulation was charged into a Bohle MC-5 (5.0 liter) bin. Magnesium stearate, NF was sieved through a No. 30 mesh screen into the MC-5 bin and the remaining granulation was charged to the bin. The mixture was blended with a Bohle LM 40 Bin blender at 25 RPM for 3 minutes and the blended granulation compressed to a target tablet weight of 262 mg using a rotary tablet press. A 7% w/w coating solution of hypromellose 2910, in purified water was prepared using a lab mixer and applied to the compressed tablets using a Vector LCDS-3 coating system to achieve an approximate 3% weight gain (˜8 mg/tablet). An 18% w/w enteric coating suspension of Acryl-Eze White (methacrylic acid co-polymer) in purified water was prepared using a lab mixer, and the suspension applied to the previously coated tablets using the Vector LCDS-3 coating system to achieve an approximate 10% weight gain (˜27 mg/tablet). The isolated tablets were white, caplet-shaped tablets approximately 0.2″×0.43″ in dimension, weighing approximately 298 mg each and containing 200 mg of compound 1 (present as the potassium salt).

One batch of 1700 capsules was prepared according to this procedure. The capsules were analyzed for identity, strength, purity, content uniformity and dissolution profiles, as described below.

### Unit Composition

| | Amount |
|---|---|
| Ingredients | (mg/tablet) |
| Compound, Milled | 200.0^{a} |
| Microcrystalline cellulose, NF | 34.0 |
| Hypromellose 2910, USP | 13.1 |
| Magnesium Stearate, NF | 1.3 |
| Total Core Tablet Weight | 262.0 |
| Hypromellose 2910, USP (First coat) | 10.0 |
| Acryl-Eze White (Enteric coat) | 27.1 |
| Total Enteric Coated Tablet Weight | 299.1 |

### Batch Composition

200 mg Tablet

| Ingredients | mg/tablet | g/1,700 Tablets |
|---|---|---|
| Active Ingredient | | |
| Compound 1, Milled | 200.0^{a} | 340.0^{a} |
| Granulation Medium | | |
| Purified Water, USP^{b} | — | q.s.^{b} |
| Excipients | | |
| Microcrystalline cellulose, NF | 34.0 | 57.8 |
| Hypromellose 2910, USP | 13.1 | 22.3 |
| Magnesium Stearate, USP | 1.3 | 2.2 |
| Total tablet core weight | 262.0 | 445.4 |
| Aqueous Coatings | | |
| Hypromellose 2910, USP | 10.0 | 17.0 |
| Acryl-Eze White | 27.1 | 46.0 |
| Total coated tablet weight | 299.1 | 508.5 |

^{a}As free acid, equivalent to 213.6 mg of potassium salt. The quantity of compound 1 used was adjusted based on the actual potency and a corresponding adjustment with microcrystalline cellulose made.
^{b}Quantity sufficient to facilitate granule formation, removed during the drying process

### Example 5B

### Identification of Compound 1 in Capsules Prepared in Example 5A

An isocratic reversed-phase HPLC method, (4.6×150 mm YMC ODS AQ, 3 μm size particles column; mobile phase of 45% 10 mM KH₂PO₄ (pH 3.0) and 55% acetonitrile) with UV detection at 220 nm, was used to confirm the identity of compound 1. The capsules were extracted with 20:80 (v/v) methanol/phosphate buffer (pH 7.4) and diluted 1:10 with methanol/water (20:80 v/v ratio). The target concentration of compound 1 for assay is 0.1 mg/mL. The identity of compound 1 was determined by comparing the retention time of the peak in the sample preparation with that of a standard preparation and showed relative retention values (Rr) 1.00±0.03 relative to reference standard, confirming the presence of compound 1.

### Example 5C

### Determination of Quantity of Compound 1 in Capsules Prepared in Example 5A

An isocratic reversed-phase HPLC method, (4.6×150 mm YMC ODS AQ, 3 μm size particles column,; mobile phase of 45% 10 mM KH₂PO₄ (pH 3.0) and 55% acetonitrile) with UV detection at 220 nm, was used to determine the amount of compound 1 present. The capsules were extracted with extracted with 20:80 (v/v) methanol/phosphate buffer (pH 7.4) and diluted 1:10 with methanol/water (20:80 v/v ratio). The target concentration of compound 1 for assay is 0.1 mg/mL.

The quantity of compound 1 present was determined by comparing sample peaks with standard preparations obtained concomitantly, and confirmed the quantity to be 102.8%, within the 90.0-110.0% limit set.

### Example 5D

### Determination of Impurities in Capsules Prepared in Example 5A

A gradient elution reversed-phase HPLC method, (4.6×150 mm YMC ODS AQ, 3 μm size particles column; mobile phase A 10 mM KH₂PO₄ (pH 3.0) and mobile phase B acetonitrile) with UV detection at 220 nm, was used to determine impurity content. The capsules were extracted with 20/80 (v/v) methanol/phosphate buffer (pH 7.4). Target concentration of compound 1 for impurities assay was 1 mg/mL. Impurities were determined by comparing the impurity peak areas in the sample preparation chromatogram to the area of the compound 1 peak in the standard preparation obtained concomitantly. Known impurities were calculated by applying the response factor of the respective impurities. The method reporting limit was 0.05%. The results of the analyses are shown in the table below.

| | Limit | Results |
|---|---|---|
| “bromo acid” impurity | NMT 1.0% | 0.07% |
| “des-bromo” impurity | NMT 1.0% | 0.41% |
| “methyl ester” | NMT 1.0% | 0.23% |
| impurity | | |
| “des-cyclopropyl” | NMT 1.5% | 0.05% |
| impurity | | |
| Unspecified | NMT 0.5% each, | RRT 0.63 = 0.06% |
| substances | report RRT | RRT 0.66 = 0.07% |
| | | RRT 0.86 = 0.06% |
| | | RRT 1.02 = 0.07% |
| | | RRT 1.06 = 0.06% |
| | | RRT 1.13 = 0.09% |
| | | RRT 1.22 = 0.05% |
| Total related | NMT 4.0% | 1.22% |
| substances | | |

### Example 5E

### Determination of Water Content in Capsules Prepared in Example 5A

Water content was determined using the USP <921>, Karl Fischer method and measured at 4.75%.

### Example 5F

### Dissolution Profile of Compound 1 from Capsules Prepared in Example 5A

Dissolution of compound 1 from the capsules was determined following the principles of the

USP <711> method A for delayed-release dosage forms, using USP Apparatus 2 at 37° C., set at 50 rpm. Acid stage was performed for 2 hours in 700 mL of dissolution medium at pH 1.2 followed by buffer stage performed in 900 mL of dissolution medium at pH 6.8. A filtered aliquot of the test dissolution medium was taken at the specified times (30, 45, 60, 90 and 100 minutes) and analyzed by HPLC using isocratic elution and UV detection at 226 nm (same chromatographic conditions as described above for content determination). Compound release was determined by comparing the peak responses of the sample chromatograms to the peak responses of the standard chromatograms obtained concomitantly and the results are shown below.

| Limit | Results | |
|---|---|---|
| Acid Stage | Mean = 0.0% | |
| Level 1: NMT 10% for | Min = 0.0% | |
| average of 6 units and no | Max = 0.0%; Pass | |
| individual greater than 25% | | |
| dissolved | | |
| Buffer Stage | 30 min | Mean = 33.9% |
| Report profile at t = 30, 45, 60, | | % RSD = |
| 90, and 100 min | | 22.0% |
| | | Min = 20.8% |
| | | Max = 43.1% |
| | 45 min | Mean = 73.4% |
| | | % RSD = |
| | | 12.5% |
| | | Min = 55.5% |
| | | Max = 80.9% |
| | 60 min | Mean = 91.2% |
| | | % RSD = 4.1% |
| | | Min = 84.3% |
| | | Max = 95.2% |
| | 90 min | Mean = 96.8% |
| | | % RSD = 2.0% |
| | | Min = 94.4% |
| | | Max = 99.4% |
| | 100 min | Mean = 101.3% |
| | | % RSD = 2.8% |
| | | Min = 97.0% |
| | | Max = 105.1% |

NMT = not more than

### III Human Clinical Studies

### Example 6

Human clinical studies using the compositions described in examples 2A, 3A, 4A, 5A were conducted as described: A multi-center, double-blind, placebo-controlled study in 48 treatment-naïve HIV-1-infected subjects, randomized 3:1 (treatment:placebo). Patients were male, 18-65 years with chronic HIV infection, but no history of AIDS-defining illness. Patients were antiretroviral treatment naïve or <14 days prior therapy and had no pre-existing RTI or PI drug resistance and no co-infection with acute HAV, chronic HBV, active HCV. Patient CD4+ cell count was ≧50 cells/mm³ for 2 cohorts, then ≧200 cells/mm³ or ≧350 cells/mm³ depending on site.

The compositions were administered over a 7-day treatment period plus one morning dose for PK purposes on day 8. (It should be noted that in some instances multiple doses of the same composition were administered, in order to achieve the required total dose of compound 1; i.e.in order to achieve a dose of 400 mg compound 1, a patient may have taken four 100 mg compositions or two 200 mg compositions, depending on the dosage form.) Four sequential dose cohorts were administered as follows:

| Capsules | EC Tablets |
|---|---|
| 400 mg BID Fasted | 800 mg QD Fed |
| 600 mg QD Fasted | 1000 mg QD Fasted |

Assessments were made as follows:
PK and tolerability Days 1-10 & 2 wks post-dose
Safety labs, immunology Days 1, 4, 9 & 2 wks post-dose
ECGs Days 1, 3, 4, 7, 9 and 2 wks post-dose
Genotype and phenotype Days 1, 9 & 2 wks post-dose
   Safety/Tolerability Observations: No serious adverse events (grade 3/4) or premature discontinuations occurred during the trail. There was no indication of CNS toxicity, no drug related rashes, no clinically significant laboratory abnormalities, no apparent effects on lipid profile, no clinically relevant ECG findings and no characteristic changes in genotypes or phenotypic susceptibility observed. Adverse events were generally mild (grade 1) with no required intervention. Patient Baseline Characteristics were determined as shown below:

| | Compound 1 | | | | |
|---|---|---|---|---|---|
| | 400 mg BID* | 600 mg QD* | 800 mg QD | 1000 mg QD* | PLACEBO |
| | N = 9 | N = 9 | N = 9 | N = 9 | N = 12 |
| Age | | | | | |
| Mean years | 35.3 | 39.9 | 31.2 | 33.0 | 36.3 |
| Race | | | | | |
| Caucasian | 7 | 9 | 7 | 7 | 8 |
| Black | 2 | — | 1 | 2 | 1 |
| Asian | — | — | — | — | — |
| Other | — | — | 1 | — | — |
| CD4 Cell | | | | | |
| Count | | | | | |
| Mean | 288.1 | 319.9 | 303.6 | 407.2 | 325.9 |
| cells/mL | | | | | |
| Viral Load | | | | | |
| Copies/ml | 31,815 | 46,845 | 40,161 | 39,852 | 32,551 |
| Range | 4880-113000 | 6060-879000 | 15900-244000 | 7520-469000 | 5730-233000 |

The Steady-State Pharmacokinetics of the various dosage forms were determined and are shown below:

| | AUC_{0-24 h} | Cₘₐₓ | Tₘₐₓ | t_{1/2} |
|---|---|---|---|---|
| | (μg · hr/mL) | (μg/mL) | (hr) | (hr) |
| 400 BID | 15.4 | 4.33 | 2.11 | 12.1 |
| MR capsule Fasted | | | | |
| 600 QD | 7.53 | 2.98 | 2.12 | 8.7 |
| MR capsule Fasted* | | | | |
| 800 QD | 9.76 | 2.76 | 5.67 | 10.8 |
| EC tablet Fed | | | | |
| 1000 QD | 16.1 | 5.72 | 3.17 | 8.5 |
| EC tablet Fasted* | | | | |

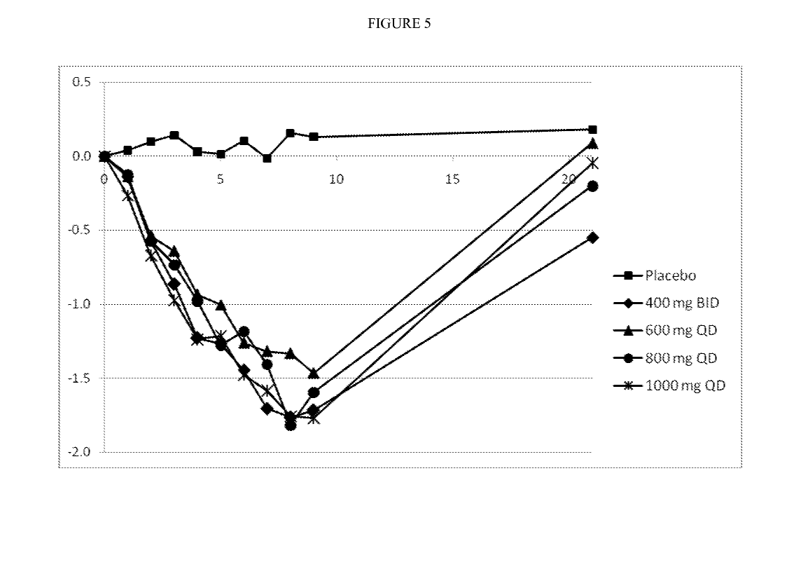

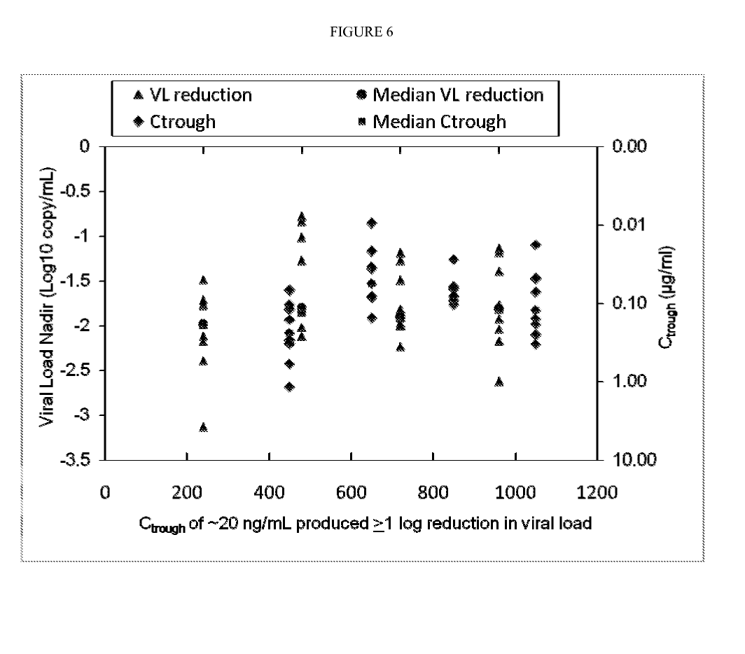

The median change in viral load for the various dosage forms was determined and the results are shown in FIGS. 5 and 6. Viral load reduction censored in 4 patients who reached 50 copies/ml LOQ of assay. Some patients started on triple therapy prior to follow-up visit

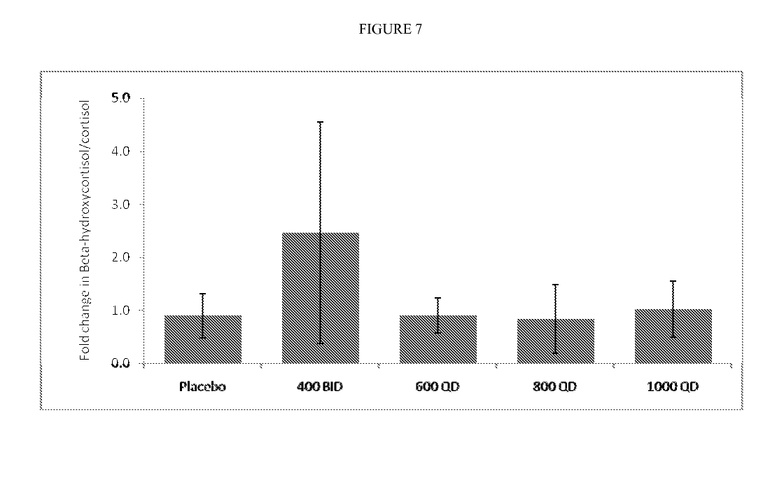

Any induction of CYP3A4 activity, measured as changes in beta-hydroxycotisol/cortisol ratio, was recorded and is shown in the table below and in FIG. 7.

| | | 600 mg | 800 mg | 1000 mg | 400 mg |
|---|---|---|---|---|---|
| | PLACEBO | QD | QD | QD | BID |
| Mean fold | 0.896 | 0.897 | 0.837 | 1.02 | 2.46 |
| change | | | | | |
| T-test p vs | | 0.994 | 0.808 | 0.556 | 0.074 |
| placebo | | | | | |

Serum uric acid levels were recorded on day 9 of the study, and are shown in the table below and graphically in FIG. 8.

| | | 600 mg | 800 mg | 1000 mg | 400 mg |
|---|---|---|---|---|---|
| | PLACEBO | QD | QD | QD | BID |
| Mean | −6 | −21 | −21 | −28 | −30 |
| serum UA | | | | | |
| reduction | | | | | |
| (%) | | | | | |
| T-test vs | | 0.0011 | 0.0010 | <0.0001 | <0.0001 |
| placebo | | | | | |

### Example 7

Additional human clinical studies were undertaken to further investigate the PK properties of the compositions described herein. The results are summarized in the table below.

| | | | | AUC_{τ} (μg · hr/mL) | | | Cₘₐₓ (μg/mL) | | |
|---|---|---|---|---|---|---|---|---|---|
| | Dose | | | Cmpd | | | Cmpd | | |
| Eg | (mg) | ¹State | N | 1 | ²M6 | ³Ratio | 1 | ²M6 | ³Ratio |
| 2A | 300 | Fa | 6 | 3.19 | 8.91 | 2.90 | 0.658 | 1.30 | 1.96 |
| | 600 | FeS | 6 | 7.34 | 19.0 | 2.85 | 2.00 | 2.52 | 1.60 |
| 2A | 300 | Fa | 8 | 3.96 | 17.6 | 5.36 | 1.44 | 2.64 | 2.93 |
| | | FeH | 8 | 3.43 | 17.9 | 5.88 | 0.467 | 1.60 | 3.76 |
| 2A | 100 | FeS | 5 | 0.670 | 2.82 | 4.04 | 0.173 | 0.264 | 2.27 |
| 2A | 300 | FeS | 6 | 3.16 | 12.4 | 5.83 | 0.667 | 1.76 | 4.39 |
| | 500 | FeS | 6 | 3.77 | 24.8 | 10.1 | 0.746 | 3.17 | 6.98 |
| 3A | 300 | Fa | 6 | 6.25 | 15.6 | 2.55 | 2.63 | 2.63 | 1.17 |
| | | FeH | 6 | 2.58 | 13.9 | 5.83 | 0.423 | 1.32 | 4.39 |
| 3A | 400 | Fa | 6 | 8.58 | 19.3 | 3.22 | 5.56 | 3.93 | 1.02 |
| 3A | 400 | Fa | 9 | 8.40 | 18.0 | 4.08 | 4.65 | 3.88 | 2.48 |
| 3A | 600 | Fa | 9 | 7.52 | 21.6 | 4.56 | 2.98 | 3.36 | 2.10 |
| 4A | 600 | Fa | 12 | 6.69 | 20.1 | 4.37 | 1.41 | 2.91 | 3.18 |
| | 600 | FeS | 12 | 5.38 | 19.6 | 6.21 | 1.19 | 2.41 | 3.87 |
| 4A | 600 | EF | 8 | 4.85 | 23.9 | 7.36 | 0.52 | 2.49 | 6.80 |
| 5A | 600 | Fa | 12 | 10.7 | 16.4 | 3.13 | 3.63 | 2.15 | 2.00 |
| | 600 | FeS | 12 | 8.31 | 22.9 | 4.74 | 2.34 | 2.65 | 2.67 |
| 5A | 600 | EF | 8 | 9.08 | 19.9 | 3.26 | 2.15 | 2.29 | 1.72 |
| 5A | 800 | FeS | 9 | 9.76 | 15.7 | 3.04 | 2.76 | 1.96 | 1.87 |
| 5A | 1000 | Fa | 9 | 16.1 | 28.3 | 3.02 | 5.72 | 3.52 | 1.37 |

¹State: Fa = Fasted; EF = Evening Fasted; FeS = Fed Standard Breakfast; FeH = Fed FDA High Fat Breakfast
²M6 is 2-(5-bromo-4-(4-cyclopropylnaphthalen-1-yl)-4H-1,2,4-triazol-3-ylthio)acetic acid-the predominant metabolite of Compound 1
³Molar Ratio M6/Compound 1

What is claimed is:

1. A pharmaceutical composition comprising: a compound or mixture of compounds of structure (1): 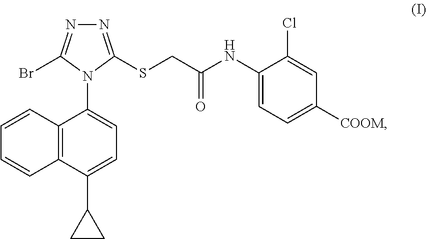
wherein M is hydrogen, sodium, potassium, calcium, or arginine; and one or more diluents; one or more binders; one or more coating agents; one or more dispersing agents; and one or more plasticizers.

2. The pharmaceutical composition of claim 1, wherein the diluent is microcrystalline cellulose, silicified microcrystalline cellulose, cellulose, lactose, compressible sugar, mannitol, calcium silicate and calcium phosphate in powder and granular forms, sodium phosphate or sodium carbonate.

3. The pharmaceutical composition of claim 1, wherein the binder is hypromellose, povidone, hydroxypropyl cellulose, hydroxyethyl cellulose or starch.

4. The pharmaceutical composition of claim 1, wherein the coating agent is a methacrylic acid based copolymers, Eudragit L30D55, Acryl-Eze, hydroxypropylmethyl cellulose acetate succinate, polyvinylacetate phthalate or cellulose acetate phthalate.

5. The pharmaceutical composition of claim 1, wherein the dispersing agent is talc, glycerol monostearate or colloidal silicon dioxide.

6. The pharmaceutical composition of claim 1, wherein the plasticizer is triethyl citrate, triacetin, dibutyl phthalate, diethyl phthalate or glycerin.

7. The pharmaceutical composition of claim 1, comprising from about 10 mg to about 1000 mg of the compound or mixture of compounds of structure (I).

8. The pharmaceutical composition of claim 1, comprising about 100 mg, or about 200 mg, or about 300 mg, or about 400 mg, or about 500 mg, or about 600 mg, or about 700 mg, or about 800 mg, or about 900 mg, or about 1000 mg of the compound or mixture of compounds of structure (I).

9. A pharmaceutical composition comprising: a compound of structure (IB) or a mixture of a compound of structure (IB) and its free acid form: 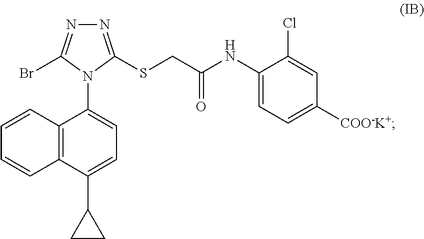
microcrystalline cellulose; hypromellose; methacrylic acid copolymer dispersion; talc; and triethyl citrate.

10. The composition of claim 9, comprising: about 214 mg of a compound of structure (IB) or a mixture of a compound of structure (IB) and its free acid form; about 35 mg of microcrystalline cellulose; about 29 mg of hypromellose; about 30 mg of methacrylic acid copolymer dispersion; about 6 mg of talc; and about 3 mg of triethyl citrate.

11. A pharmaceutical composition comprising: from about 60% to about 90% by weight of a compound of structure (IB) or a mixture of a compound of structure (IB) and its free acid form: 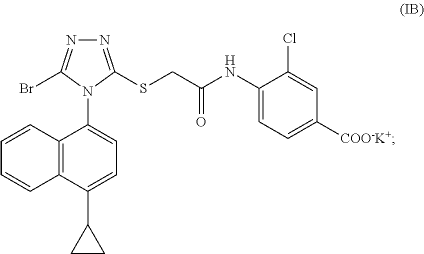
from about 5% to about 15% by weight of microcrystalline cellulose; from about 5% to about 15% by weight of hypromellose; from about 5% to about 15% by weight of methacrylic acid copolymer dispersion; from about 0.5% to about 5% by weight of talc; and from about 0.1% to about 3% by weight of triethyl citrate.

12. A pharmaceutical composition comprising: a compound of structure (IB) or a mixture of a compound of structure (IB) and its free acid form: 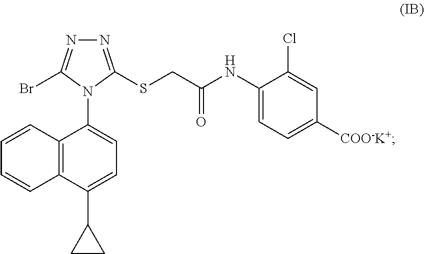
microcrystalline cellulose; and hypromellose; wherein the composition is in the form of granules.

13. A pharmaceutical composition comprising: about 214 mg of a compound of structure (TB) or a mixture of a compound of structure (IB) and its free acid form: 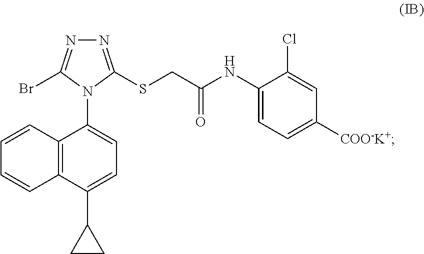
about 35 mg of microcrystalline cellulose; about 13.5 mg of hypromellose; wherein the composition is in the form of granules which do not pass through a 40 mesh screen; and wherein the granules are coated with about 15.3 mg hypromellose; and wherein the coated granules are further coated with a composition comprising: about 30.4 mg of methacrylic acid copolymer dispersion; about 6.1 mg of talc; and about 3.0 mg of triethyl citrate.

14. The composition of claim 1, wherein the in vitro dissolution rate as measured using United States Pharmacopoeia Apparatus 1 operating at 75 rpm in 900 mL water at 37° C. is not less than about 80% of the compound of structure (IB) is released within 30 minutes.

15. A method for treating or preventing HIV infection, comprising administering to a subject in need thereof an effective amount of a composition according to claim 1.

16. A method for preventing infection with an immunodeficiency virus in a subject, treating infection with an immunodeficiency virus in a subject, preventing acquired immunodeficiency syndrome (AIDS) in a subject, treating acquired immunodeficiency syndrome (AIDS) in a subject, preventing an AIDS-related complex (ARC) in a subject or treating an AIDS-related complex (ARC) in a subject, comprising administering to the subject an effective amount of a composition according to claim 1.

17. A method of inhibiting an immunodeficiency virus comprising contacting said immunodeficiency virus with a composition according to claim 1.
